(19) United States
(12) Patent Application Publication
(10) Pub. No.: US 2003/0082229 A1
(43) Pub. Date: May 1, 2003

(54) Parenteral chlorambucil for treatment of malignant and autoimmune disease and methods of use

(75) Inventors: Borie S. Anderson, Houston, TX (US); Taraneh K. Sadeghi, Houston, TX (US)

Correspondence Address: BAKER BOTTS L.L.P., PATENT DEPARTMENT, 98 SAN JACINTO BLVD., SUITE 1500, AUSTIN, TX, 78701-4039, US

(73) Assignee: Board of Regents, The University of Texas Systems

(21) Appl. No.: 10/284,791

(22) Filed: Oct. 31, 2002

Related U.S. Application Data
(60) Provisional application No. 60/344,530, filed on Nov. 1, 2001.
(60) Provisional application No. 60/344,580, filed on Nov. 1, 2001.
(60) Provisional application No. 60/348,214, filed on Nov. 9, 2001.

(51) Int. Cl. A61K 31/195; A61K 9/127

(52) U.S. Cl. 424/450; 514/567

(57) ABSTRACT

A parenteral composition including a first solvent, made of an alcohol and a lipid, and a water soluble agent such as chlorambucil dissolved in the first solvent. The composition may be further diluted with an infusion fluid, such as normal saline, before infusion into a patient. The chlorambucil composition is useful for the treatment and suppression of malignant and autoimmune disease.

### CROSS REFERENCE TO RELATED APPLICATIONS

This application claims the benefit, under 35 U.S.C. §119(e), of U.S. Provisional Patent Application Ser. No. 60/344,530, filed Nov. 1, 2001 entitled “Parenteral Chlorambucil for Treatment of Malignant and Autoimmune Disease,” U.S. Provisional Patent Application Ser. No. 60/344,580, filed Nov. 1, 2001 entitled “Parenteral Chlorambucil for Treatment of Malignant and Autoimmune Disease,” U.S. Provisional Patent Application Ser. No. 60/348,214, filed Nov. 9, 2001 entitled “Parenteral Chlorambucil for Treatment of Malignant and Autoimmune Disease and Methods of Use,” all of which applications are hereby incorporated herein by reference in their entirety.

### TECHNICAL FIELD

The present invention is related generally to a therapeutic composition for parenteral use and treatments for malignancies, autoimmune disease and other diseases. It relates specifically to a composition and method for parenteral use of chlorambucil in the treatment of malignancies and autoimmune disease.

### BACKGROUND OF THE INVENTION

The chemotherapeutic agent Chlorambucil (CLB; 4-[Bis(2-chloroethyl)amino]benzenebutanoic acid; C₁₄H₁₉Cl₂NO₂) over the last several decades has earned an impressive reputation for its efficacy against chronic lymphocytic leukemia (CLL) and low-grade lymphomas (Rai K R: Chronic Lymphocytic Leukemia. In: Hoffman R, Benz E J, Jr., Shattil S J, Furie B, Cohen H J (Eds). HEMATOLOGY. BASIC PRINCIPLES AND PRACTICE, Churchill Livingstone Inc. New York, Philadelphia, pp 990-1001, 1991; Bierman P J, Vose J M, Armitage J O: Clinical manifestations staging and treatment of non-Hodgkin's Lymphomas. In: Hoffman R, Benz E J, Jr., Shattil S J, Furie B, Cohen H J (Eds). HEMATOLOGY BASIC PRINCIPLES AND PRACTICE, Churchill Livingstone Inc. New York, Philadelphia, pp 963-982, 1991; Cadman E, Drisbane F, Waldron J A, et al. High dose chlorambucil: effective therapy for rapid remission in nodular lymphocytic poorly differentiated lymphoma. CANCER, 50:1037, 1982; Keller J W, Knospe W H, Raney M, et al: Treatment of chronic lymphocytic leukemia using chlorambucil and prednisone with or without cycle-active consolidation chemotherapy. CANCER 58:1185-1192, 1986; Binet J L, et al. A randomized clinical trial of chlorambucil versus COP in stage B chronic lymphocytic leukemia. BLOOD. 75:7, 1422-1425, 1990; Binet J L, et al. Is the CHOP regimen a good treatment for advanced CLL? Results from two randomized clinical trials. LEUKEMIA AND LYMPHOMA. 13:449-456, 1994; Jaksic B, Brugiatelli M, Krc I, et al. High dose chlorambucil versus Binet's modified cyclophosphamide, doxorubicin, vincristine, and prednisone regimen in the treatment of patients with advanced B-cell chronic lymphocytic leukemia. CANCER, 79:11, 2107-2114, 1997; Wilhelm M, Tony H P, Rueckle-Lanz H, Wilms K. First-line therapy of advanced chronic lymphocytic leukemia. LEUKEMIA 11:2, S14-S18, 1997; Dighiero G, Maloum K, Desablens B, et al. Chlorambucil in indolent chronic lymphocytic leukemia. THE NEW ENGLAND JOURNAL OF MEDICINE. 338:21, 1506-1514, 1998; Knospe W H, Loeb B, Jr., Huguley C M, Jr., Bi-weekly chlorambucil treatment of chronic lymphocytic leukemia. CANCER, 33:555-562, 1974; Sawitsky A, Rai K R, Glidewell O, et al. Comparison of daily versus intermittent chlorambucil and prednisone therapy in the treatment of patients with chronic lymphocytic leukemia. BLOOD, 50:6, 1049-1059, 1997; Stolzenbach G, Garbrecht M. Intermittent combination chemotherapy with chlorambucil and prednisone of low-grade malignant non-Hodgkin's Lymphomas according to the Kiel classification. J. CANCER RES. CLIN. ONCOL. 93, 189-194, 1979).

Its most widespread use has been in the treatment of CLL and indolent, low-grade lymphomas, although it has also demonstrated activity in Hodgkin's disease, myeloproliferative disorders and advanced autoimmune diseases (Dady P J, McElwain T J, Auston D E, et al: Five years' experience with ChlVPP effective low-toxicity combination chemotherapy for Hodgkin's Disease. BRIT. J. CANCER. 45:851-859, 1982; Berk P D, Goldberg J N, Donovan P B, et al: Therapeutic recommendations in polycythemia vera based on polycythemia vera study group protocols. SEMIN. HEMATOL. 23: 132-143, 1986; Ponticelli C, Zuchelli P, Passerini P, et al: A 10-year follow-up of a randomized study with methylprednisolone and chlorambucil in membranous nephropathy. KIDNEY INT, 48:1600-4, 1995; Mathieson P W, Turner A N, Maidment C G, et al: Prednisolone and chlorambucil treatment in idiopathic membranous nephropathy with deteriorating renal function. LANCET, 2:869-72, 1988; Warwick G L, Geddes C G, Boulton-Jones J M: Prednisolone and chlorambucil therapy for idiopathic membranous nephropathy with progressive renal failure. QJ MED, 87:223-9 1994).

With the introduction of combination chemotherapy and nucleoside analogs, e.g., fludarabine (Keating M J, Kantarjian H, Talpaz M, et al: Fludarabine: a new agent with major activity against chronic lymphocytic leukemia. BLOOD 74:19-25, 1989; Keating M J, Kantarjian H, O'Brien S, et al: Fludarabine: A new agent with marked cytoreductive activity in untreated chronic lymphocytic leukemia. J. CLIN. ONCOL. 9:44-49, 1991; Hiddermann W, Rottmann R, Wormann B, et al: Treatment of advanced chronic lymphocytic leukemia by fludarabine. Results of a clinical phase II study. ANN. HEMATOL. 63:1-4, 1991), CLB was thought to be inferior for long-term disease control.

However, more recent studies comparing different treatment modalities suggest that CLB as single agent therapy, or in combination with steroids, is equivalent to fludarabine in terms of overall patient survival (Binet J L, et al. A randomized clinical trial of chlorambucil versus COP in stage B chronic lymphocytic leukemia. BLOOD. 75:7, 1422-1425, 1990; Binet J L, et al. Is the CHOP regimen a good treatment for advanced CLL? Results from two randomized clinical trials. LEUKEMIA AND LYMPHOMA. 13:449-456, 1994; Rai K R, Peterson B, Elias L, et al: A randomized comparison of fludarabine and chlorambucil for patients with previously untreated chronic lymphocytic leukemia, A CALGB, SWOG, CTG-NCI-C and ECOG Intergroup Study. BLOOD 88: 1451a, 1996; Jaksic B, Brugiatelli M, Suciu C, et al: Randomized phase II study in untreated B-cell chronic lymphocytic leukemia comparing Fludarabine versus high dose continuous chlorambucil. BLOOD 92:421a, 1998).

Recent randomized evaluations of fludarabine versus combination chemotherapy have also failed to confirm the previously perceived superiority of fludarabine in the treatment of CLL (Johnson S, Smith A G, Loffler H: Multicentre prospective randomized trial of fludarabine versus cyclophosphamide, doxorubicin, and prednsione (CAP) for treatment of advanced-stage chronic lymphocytic leukemia. The French Cooperative Group on CLL. LANCET 347: 1432-1438, 1996; Leporrier M, Chevret S, Cazin B, et al: Randomized comparison of fludarabine, CAP and CHOP in 695 previously untreated stage B and C chronic lymphocytic leukemia. Early stopping of the CAP accrual. BLOOD 90: 2357a, 1997).

These findings have revived the clinical interest in CLB, whose main clinical advantages reside in its low price and its ease of use (as an oral preparation). Early studies in different malignancies utilizing several different dosing schedules, including fairly intensive programs, suggested that the CLB activity spectrum primarily includes the lymphoid malignancies. Such studies further indicate that once-a-month courses of up to 100 mg/m² body surface area may be clinically equivalent to, and more convenient than, daily oral administration (Rai K R: Chronic Lymphocytic Leukemia. In: Hoffman R, Benz E J, Jr., Shattil S J, Furie B, Cohen H J (Eds). HEMATOLOGY. BASIC PRINCIPLES AND PRACTICE, Churchill Livingstone Inc. New York, Philadelphia, pp 990-1001, 1991; Jaksic B, Brugiatelli M, Krc I, et al. High dose chlorambucil versus Binet's modified cyclophosphamide, doxorubicin, vincristine, and prednisone regimen in the treatment of patients with advanced B-cell chronic lymphocytic leukemia. CANCER, 79:11, 2107-2114, 1997; Knospe W H, Loeb B, Jr., Huguley C M, Jr., Bi-weekly chlorambucil treatment of chronic lymphocytic leukemia. CANCER, 33:555-562, 1974; Sawitsky A, Rai K R, Glidewell O, et al. Comparison of daily versus intermittent chlorambucil and prednisone therapy in the treatment of patients with chronic lymphocytic leukemia. BLOOD, 50:6, 1049-1059, 1997).

CLB is assumed to be rapidly absorbed from the intestinal tract. However, accurate data regarding intestinal absorption are not available, nor are data regarding possible inter-individual variations in hepatic first-pass metabolism and overall bioavailability of CLB due to the absence of an intravenous (IV) reference formulation. The instability of CLB in aqueous solution has prevented the development of a parenteral CLB formulation that could be used either for routine clinical administration or for pharmacological investigations. This lack of an IV preparation has prevented the development of optimal administration schedules, and therefore hampered the clinical use of CLB.

Recent pharmacokinetic data for the drug busulfan has provided information relating increased risks for: 1) serious clinical toxicity; 2) marrow graft rejection; and 3) leukemic relapse when oral busulfan was used in high-dose conditioning therapy with allogeneic hematopoietic stem cell transplantation. Such findings encouraged the development of an IV busulfan formulation, which is now replacing the standard oral preparation in many intensive treatment programs for myeloid leukemia.

Like busulfan, CLB is useful in treating lymphoid and myeloid hematological malignancies. The two drugs have chemical similarities, including their side effects (e.g., toxicity, such as grand mal seizures and interstitial pneumonitis). Just as IV busulfan has been found to be therapeutically valuable, an IV formulation of CLB would also be extremely useful.

Thus, there exists a need for chlorambucil formulations that are suitable for parenteral administration such as intravascular (including intravenous and intra-arterial) and intrathecal administration, and methods for their use.

### SUMMARY OF THE INVENTION

The present invention provides pharmaceutically stable and parenterally-acceptable, novel formulations of CLB that can be utilized for the intravascular, intrathecal or other parenteral treatment of malignant, autoimmune diseases and other diseases in man and animals. The invention relates to pharmaceutical formulations, and more specifically, to parenteral formulations of chlorambucil (CLB). Parenteral formulations of the invention are useful for the treatment and/or suppression of malignant or autoimmune diseases. The parenteral formulations avoid the undesirable erratic bioavailability of oral preparations. They can also be used for the treatment of local disease in sanctuary sites, e.g. by the intrathecal route to treat central nervous system disease.

The formulations of the invention are based on the cosolvency approach. This pharmaceutical principle is utilized in the pharmaceutical industry and approved by the U.S. Food and Drug Administration (FDA). Exemplary cosolvent compositions of the invention are pharmaceutically acceptable and nontoxic. Additional examples are also stable at room temperature.

Formulations according to the invention can be mixed with clinically acceptable aqueous parenteral infusion fluids, such as normal saline or dextrose in water, as final solvent(s).

One embodiment of the invention is directed to a chlorambucil-containing composition for parenteral use including chlorambucil and a first solvent including an alcohol and a lipid, such as ethanol (EtOH) and soybean oil. The chlorambucil is dissolved in the first solvent. Prior to administration, the composition may be diluted with a second solvent comprising an aqueous infusion fluid.

The novel solvent vehicles of the invention are not limited to chlorambucil, but may also be used to facilitate parenteral administration of other water-insoluble drugs. Water-insoluble includes drugs that are poorly soluble in water. Accordingly, another embodiment of the invention relates to a composition for parenteral use which includes a water-insoluble/lipophilic pharmaceutically active agent and a first solvent where the first solvent includes an alcohol and a lipid. Alternately, the first solvent may be an acid and a lipid. The agent is dissolved in the first solvent. The composition optionally further includes a second solvent including an aqueous infusion fluid.

The invention also includes a method of preparing a water-insoluble/lipophilic pharmaceutically active agent for parenteral use by the steps of: providing an aqueous liquid emulsion; lyophilizing the aqueous lipid emulsion; emulsifying the lyophilized emulsion in alcohol to produce a primary diluent; and dissolving the pharmaceutically active agent in the primary diluent to produce a stock formulation. In one embodiment, the lipid is soybean oil, the alcohol is ethanol (EtOH) and/or the agent is chlorambucil. The method may further include the step of mixing the stock formulation with a second diluent, such as an aqueous infusion fluid.

The invention also includes a method for treating a disease sensitive or responsive to chlorambucil by parenterally administering a therapeutically effective amount of a chlorambucil composition to the patient. The chlorambucil composition includes chlorambucil and a first solvent, including an alcohol and a lipid. The chlorambucil is dissolved in the first solvent. Certain embodiments may also include a second solvent, including an aqueous infusion fluid.

Still another embodiment of the invention is directed to a method for parenterally administering chlorambucil to a patient by providing an aqueous lipid emulsion; lyophilizing the aqueous lipid emulsion; emulsifying the lyophilized emulsion in alcohol to produce a primary diluent; dissolving the chlorambucil in the primary diluent to produce a stock formulation; mixing the stock formulation with a second diluent to form an infusion fluid; and administering the infusion fluid to the patient. In exemplary embodiments the lipid is soybean oil and the alcohol is ethanol.

### DESCRIPTION OF THE FIGURES

The following figures form part of the present specification and are included to further demonstrate certain aspects of the present invention. The invention may be better understood by reference to one or more of these drawings in combination with the detailed description of specific embodiments presented herein.

FIG. 1 is a graph showing the stability of chlorambucil at room temperature in the formulation of EtOH/lipid (i.e., prototype EtOH/lipid solvent vehicle) containing CLB diluted with NS to 1 mg/ml (-▪-), and to 5 mg/ml (-□-), respectively, according to an embodiment of the present invention. Two different lots of CLB were solubilized and tested in parallel. The x-axis represents the time in hours, and the y-axis represents the area under the curve (AUC, a term used to denote the actual measured area of a peak in a chromatogram), in this graph, expressed as a percent of the area under the curve.

FIG. 2 is the standard curve of chlorambucil concentration vs. area under the curve for a high-pressure liquid chromatography (HPLC) assay used in stability studies. The x-axis shows concentration in μg/ml, and the y-axis shows the AUC. An analogous standard curve was prepared for a pharmacology study.

FIG. 3 depicts two chromatograms obtained from an HPLC assay in stability studies of an embodiment. In 3(a), a Whatman C-18 EQC 10 μL 125 A μBondapak column was used. The injected sample volume (100 μg/ml) was 200 μl. In 3(b), a C₈Symmetry™ column was used and the injected volume was 20 μl (injected concentration of 100 μg/ml).

FIG. 4 is a graph showing the hemolytic potential of a formulation of EtOH/lipid (prototype EtOH/lipid solvent vehicle) in normal saline (NS) with CLB (-▪-), and the same formulation without CLB (-□-), referred to as “vehicle”, according to an embodiment of the present invention. The x-axis shows the concentration in μg/ml. The y-axis shows the percent hemolysis.

FIG. 5 is a graph depicting the cytotoxic activity of CLB in an EtOH/lipid/NS formulation according to an embodiment of the present invention against the human cell lines KBM-7/B5 (-▪-) and HL-60 (-▴-) assessed with in vitro clonogenic assay with prolonged drug exposure. The x-axis shows the concentration in μg/ml; the y-axis shows the percent survival.

FIG. 6 shows chromatograms of plasma samples extracted with OASIS extraction cartridges and then subjected to HPLC. FIG. 6(a) shows a blank plasma sample, FIG. 6(b) shows a plasma sample spiked with CLB in a formulation according to an embodiment of the present invention (prototype EtOH/lipid solvent vehicle) to 100 μg/ml. FIG. 6(c) shows a chromatogram from a pharmacology study, where a beagle was injected with CLB, formulated according to an embodiment of the present invention, at 7.5 mg/kg. The chromatogram was from a sample drawn 30 minutes after drug injection.

FIG. 7 is a graph showing the change in plasma concentration over time of 7.5 mg/kg CLB, formulated according to an embodiment of the present invention, injected into a beagle dog. The x-axis shows the time after dose in hours. The y-axis shows the concentration of CLB in μg/ml plasma.

### DETAILED DESCRIPTION OF THE PREFERRED EMBODIMENTS

The present invention is directed to novel formulations containing chlorambucil (CLB) or other drugs that are poorly-water-soluble or water-insoluble that may be administered parenterally. The invention provides for solubilized chlorambucil (CLB) or other drugs that are water-insoluble drugs (including poorly-water-soluble drugs) in complex, pharmaceutically-acceptable vehicles such that the dissolved drug remains physically and chemically stable. The invention allows for parenteral administration of the drug in doses necessary to obtain significant cytotoxic and immunosuppressive effects in humans and animals without undue toxicity from the solvent vehicle. Additional beneficial effects without undue cytotoxicity may also be obtained. Exemplary embodiments of the invention allow for the intravascular or intrathecal administration of CLB solubilized in alternative formulations to increase the clinical safety of drug administration. As a result, an improved control of malignant and autoimmune diseases that are sensitive to this agent may be achieved.

Although CLB is discussed herein as an example, other water-insoluble drugs (including poorly-water-soluble drugs) for parenteral use are included within the scope of the present invention. Such drugs may be formulated according to the methods described herein. Some drugs may be solubilized using acid or lipid. Durgs soluble in ethanol and lipid or alcohol and soybean oil may be prepared using a methodology very close to that of the present invention. Variations in solubilizing agents will be apparent to one skilled in the art and will depend inter alia on the chemical structure of the drug. Such formulations of other drugs may be useful to achieve treatments associated with such drugs. These treatments may differ from the anticancer or immunosuppressive effects associated with CLB.

CLB as an orally administered anticancer agent has previously been extensively investigated in humans; the drug has well documented cytotoxic and immunosuppressive properties as reported in both clinical and experimental settings. However, prior to the present invention, an acceptable parenteral formulation of CLB has not been available. A parenteral formulation of CLB would be useful to evaluate CLB as therapy for systemic malignant and autoimmune disorders as well as when profound immunosuppression is desirable, such as that required for allogeneic hematopoietic stem cell transplantation, since it gives more complete control of the delivery/pharmacokinetics of the drug. Unfortunately, CLB is a poorly water-soluble DNA-alkylating agent with exceedingly low solubility in physiologically acceptable aqueous solvents that would be compatible with human intravascular administration. Prior to the present invention, the only available administration form of CLB has been an oral preparation.

One embodiment of the present invention, based on the principle of cosolvency, uses novel composite diluent vehicles to solubilize CLB without affecting its cytotoxic properties. Further, the example solvents are, in the proposed concentrations and total doses used, nontoxic and safe for human and other mammalian routes of administration, including intravenous, intra-arterial and intrathecal.

As discussed in the Examples below, novel vehicles have been discovered which achieve the stable, pharmaceutically acceptable solubilization of chlorambucil, thereby making it safe to administer this drug intravascularly. A sensitive and specific HPLC assay was initially established, which allowed the reproducible quantitation of CLB in concentrations as low as 5-10 ng/ml. In parallel, extraction techniques were developed to recover CLB from blood plasma after intravenous (IV) administration. Stability studies of the newly formulated vehicles were then initiated to help identify the best formulation for in vitro studies of hemolytic potential and cytotoxic activity. Human leukemic cell lines HL-60 (Gallagher R, Collins S, Trujillo, J. et al: Characterization of the continuously differentiating myeloid cell line (HL-60) from a patient with acute promyelocytic leukemia. BLOOD 54:254-68, 1979), and KBM-7/B5 (Andersson B S, Beran M, Pathak S, et al: Ph-positive chronic leukemia with near-haploid conversion in vivo and establishment of a continuously growing cell line with similar cytogenetic pattern. CANCER GENETICS AND CYTOGENETICS 24:335-43, 1987; Andersson B S, Collins V P, Kurzrock R, et al: KBM-7; Human myeloid leukemia cell line with double Philadelphia chromosomes but lacking normal BCR and c-ABL transcripts. LEUKEMIA, 9:2100-2108, 1995) were used as paradigms for malignant disease. One of the stable new formulations (CLB dissolved in the prototype EtOH/lipid solvent vehicle and diluted with normal saline (NS)) was injected IV in rats at 5.0 mg/kg body weight (BW), and in beagles at 7.5 mg/kg BW. The results illustrate that cytotoxic/immunosuppressive CLB concentrations can be obtained after IV injection of this novel formulation in animals. Further, the new formulation yielded plasma drug concentrations and areas under the plasma concentration vs. time curves that clearly were in the cytotoxic range, demonstrating that exemplary formulations of the invention may be used for intravascular treatment of malignant and advanced autoimmune disorders in animals including humans.

As shown in the Examples, CLB was successfully formulated for intravascular use using nontoxic solvent systems based on the principle of cosolvency (Spiegel A. J. Noseworthy M. N.: Use of nonaqueous solvents in parenteral products. J. PHARM. SCI. 52:917-927, 1963; Yalkowsky S. H., Roseman T. J.: Solubilization of drugs by cosolvents. In: Yalkowsky S. H. (Ed.): TECHNIQUES OF SOLUBILIZATION OF DRUGS. Pp. 91-134. Marcel Dekker Inc., New York, N.Y. 1981). Using non-toxic primary solvent vehicles mixed with clinically acceptable infusion fluids, formulations stable for more than 12 hours at room temperature were produced. CLB formulations (e.g., prior to the addition of the secondary/final diluent) are stable for more than one week at room temperature, are simple to handle, and provide reliable and easily controlled, consistent dose administration.

In one embodiment of the invention, CLB is dissolved using ethanol in combination with non-aqueous emulsified soybean oil as the primary vehicle or solvent. The lipid emulsion may be preferably first lyophilized and emulsified in ethanol for use as a primary composite diluent, a procedure that has not been previously documented. The CLB is then dissolved in the ethanol/soybean emulsion (freeze-dried lipid). These solvents are miscible in secondary/final aqueous solvents, e.g., routinely available aqueous infusion fluids such as 0.9% sodium chloride (NS), and dextrose in water. Such terminal solvents/infusion fluids are typical examples of vehicles used to solubilize pharmacologically active agents for human administration, alone or in combination with other drugs. Prior to IV administration, the EtOH/lipid/CLB is then mixed with the secondary/final diluent.

Chlorambucil is very lipophilic, and the use of an ethanol/lipid emulsion dissolves it and stabilizes the agent in the lipid phase for further dilution in the aqueous diluent. The stability of the formulation permits infusions in excess of 24 hours without loss of drug activity due to physical precipitation or chemical degradation. Pharmaceutically acceptable grades of emulsified soybean oil for parenteral administration in humans are readily available. Their aqueous counterparts (e.g., “Intralipid™” (Pharmacia, Peapack, N.J.) and “Liposyn™” (Abbott, Abbot Park, Ill.) have long been approved for routine parenteral nutrition.

As shown in the Examples, the described EtOH/lipid-based vehicles were successfully used to dissolve CLB at concentrations ranging from 0.1 to at least 100 mg/ml. This range is broad enough to cover the administration of doses necessary to yield cytotoxic concentrations in vivo to treat malignancies sensitive to this drug. Similarly, this range is sufficient to achieve effective immunosuppression in patients with autoimmune disorders and in those undergoing conditioning therapy preceding hematopoietic stem cell transplantation.

Data obtained in experimental animals demonstrate that stable CLB formulation, prepared according to the present invention, will allow parenteral treatment of systemic malignant and autoimmune diseases. This preparation by definition consistently provides 100% drug bioavailability, and it allows circumvention of hepatic first-pass metabolism. After a brief IV injection, the plasma CLB concentrations clearly reach, and for extended time remain in, the cytotoxic range as established by the in vitro studies of its cytotoxic activity against human malignant cell lines.

A variety of biological and chemical methods were used to demonstrate that exemplary CLB formulations are stable at 100 mg/ml for several days at room temperature (RT). As shown in the Examples, one such formulation (EtOH/lipid/CLB) is stable at 100 mg/ml for at least 7 days, and retains full cytotoxic activity when assayed in vitro against the two human leukemic cell lines. Commercially available CLB was dissolved in acetone and used as a reference solvent system (acetone/water) for the in vitro cytotoxicity assay. Further, the EtOH/lipid vehicle is nontoxic as assayed in a hemolysis assay. One of the novel formulations was used to show that cytocidal CLB concentrations are maintained for several hours in both rodent and beagle models after IV injection of up to 7.5 mg/kg body weight.

Although an exemplary embodiment of the invention uses EtOH and soybean oil, in normal saline, other solvent vehicles that are non-toxic and safe for human administration may also be used. One solvent, EtOH, that has previously been used to solubilize various pharmacologically active agents for administration in man, is routinely used as an antidote for methanol poisoning (Robertson R: Common poisonings. In: Wyngarden J B, and Smith L H (Eds.) CECIL'S TEXTBOOK OF MEDICINE. W B Saunders Company, Philadelphia, Pa. 140-145, 1988; Murdoch Ritchie J: The aliphatic alcohols. In: Goodman Gilman A, Goodman L S, Rall T W, Murad F (Eds.). GOODMAN AND GILMAN'S THE PHARMACOLOGICAL BASIS OF THERAPEUTICS. 7th Ed. Macmillan Publishing Company, New York, N.Y. 372-386, 1985), and has for many years been used to interrupt premature labor (Symposium (Various Authors; Creasy R K, Ed.) Preterm Parturition. SEMIN. PERINATOL. 5: 191-302, 1981). Dimethylsulfoxide (DMSO) has been extensively used in clinical medicine, most widely as a cryoprotective agent for preservation of bone marrow and blood progenitor cells for hematopoietic stem cell transplantation (Davis J M, Rowley S D: Autologous bone marrow graft processing. In: Sacher R A, McCarthy L J, Smith Siblinga C (Eds.).: PROCESSING OF BONE MARROW FOR TRANSPLANTATION. American Association of Blood Banks, Arlington Va., pp.41-62, 1990; Gorin N C: Cryopreservation and storage of stem cells. In: Areman E M, Deeg J H, Sacher RA. BONE MARROW AND STEM CELL PROCESSING: A MANUAL OF CURRENT TECHNIQUES. F. A Davis Company, Philadelphia Pa., pp. 292-362, 1992).

No serious clinical adverse effects have been experienced from the human use of these diluents. As alternatives to EtOH, one could also use an organic acid such as acetic acid to drastically change the pH and thereby allow solubilization of the pharmacologically active agent. The clinical use of normal saline, dextrose in water (5-7%), and aqueous lipid emulsions are established, routine means to correct fluid and electrolyte balance and to supply parenteral nutrition. Normal saline and dextrose in water are also extensively used to dilute various medications for IV use. The aqueous lipid emulsion has not yet found widespread use as a pharmaceutical diluent, but this use has been suggested (Fortner C L, Grove W R, Bowie D, Walker M D: Fat emulsion vehicle for intravenous administration of an aqueous insoluble drug. AM. J. HOSP. PHARM. 32:582-84, 1975). There may be circumstances where mixing ethanol(EtOH) or lipid with protein, such as albumin, may be desirable or where a protein-containing solution alone, such as albumin, may be used as a diluent.

The compositions of the invention have a number of uses. As noted, preferred formulations of the invention are particularly useful in the treatment of malignancies and autoimmune diseases in man and animals. Certain malignancies, most notably the lymphoid malignancies such as chronic lymphocytic leukemia, low-grade lymphomas, and Hodgkin's Disease, may be controlled by CLB for prolonged time periods. The nontoxic, pharmaceutically acceptable, water miscible, intravascular CLB preferred formulations of the invention eliminate the risk of treatment failure from unpredictable and erratic intestinal absorption and first-pass liver elimination/metabolism that to varying degrees characterize administration of the oral standard preparation. The potential benefits of the intravascular formulation include fewer side effects than with the oral drug, since intravascular administration gives complete control of the bioavailability and pharmacokinetics of the drug.

The novel composite solvent vehicle(s) of the invention may also be used to investigate different administration schedules (e.g., prolonged IV infusions) in order to optimize treatment outcome for CLB-based therapy. The invention makes it possible to investigate the benefits of different dose schedules of CLB against various systemic diseases without the confounding adverse effects from unpredictable intestinal drug absorption and hepatic first-pass effects that in an arbitrary fashion influence the metabolism of CLB after oral administration. The availability of a parenteral preparation is of particular interest when dose-intensive schedules are contemplated. In this particular situation, a firm control of both drug bioavailability and pharmacokinetics are of utmost importance to ensure the patient's safety through control of a drug's clinical side effects, while maximizing the chance for disease control.

Further, the stability of the new formulations makes them particularly suited for the evaluation of different administration schedules, including that of prolonged infusions, further realizing the outstanding therapeutic potential of CLB. The stable solubilization also allows for the intrathecal application of CLB for the treatment of leptomeningeal malignant spread.

Finally, as will be clear to those skilled in the art, the solvent vehicles of the invention are not limited to use with CLB, and can be utilized in an analogous fashion to make parenteral systems for other poorly water-soluble biologically-active agents and drugs. These are exemplified by, but not limited to, antibiotics and antineoplastic agents. Such other drugs and agents include, but are not limited to, cytotoxic agents such as epipodophyllotoxin derivatives, taxanes, Bleomycin, anthracyclines, as well as platinum compounds. They also include antibiotics, such as the poorly water-soluble polyenes (e.g., Amphotericin B and Natamycin) and azole derivatives as well as antibacterial agents, (e.g., polymyxin B), anti-viral drugs and tranquilizing/anesthetic drugs such as benzodiazepines and anti-psychotic agents. Thus, in a broader sense, the present invention provides a method to safely solubilize and administer many poorly water-soluble, pharmacologically-active agents, in addition to CLB.

Accordingly, one embodiment of the invention is directed to a chlorambucil-containing composition for parenteral use including chlorambucil and a first solvent including an alcohol and a lipid. The chlorambucil is dissolved in the first solvent. Prior to administration, the composition may be diluted with a second solvent including an aqueous infusion fluid.

In further variations of this embodiment, the first solvent is an emulsion of an alcohol and a lipid, such as ethanol and soybean oil. The soybean oil is in the form of an aqueous liquid emulsion, and may be a freeze-dried, aqueous soybean oil emulsion. Sources of soybean oil include, but are not limited to, FDA-approved Intralipid™ or Liposyn™.

The ethanol may constitute between 5 and 99% of the first solvent and the soybean oil may constitute between 1 and 95% of the first solvent. In a specific embodiment, the ethanol constitutes between 90 and 99% of the first solvent and the soybean oil constitutes between 1 and 10% of the first solvent.

The invention is not limited to ethanol and soybean oil as the first solvent. Other solvents, such as organic acids, particularly acetic acid, may be used to substantially alter the pH. Useful infusion fluids include, but are not limited to, normal saline and dextrose in water. Alternately, the infusion fluid may be a lipid-based infusion emulsion fluid such as those used for parenteral nutrition. There may be circumstances where mixing lipid with protein, such as albumin, may be desirable or where a protein-containing solution alone, such as albumin, may be used as a diluent.

Prior to dilution with the infusion fluid, one composition of the present invention may include between 1 and 100 mg/ml of chlorambucil and, more specifically between 10 mg/ml to 25 mg/ml of chlorambucil. Such an undiluted composition may be stable for at least 7 days at room temperature.

In one embodiment, the second solvent is normal saline and the composition includes between 1 mg/ml and 5 mg/ml of chlorambucil after dilution in the second solvent. This diluted composition may be stable for at least 12 hours at room temperature.

The novel solvent vehicles of the invention are not limited to chlorambucil, but may also be used to facilitate parenteral administration of other drugs that are water-insoluble or poorly-water-soluble. As noted, such drugs include, but are not limited to, cytotoxic agents such as epipodophyllotoxin derivatives, taxanes, Bleomycin, anthracyclines, as well as platinum compounds. They also include antibiotics, such as the poorly water-soluble polyenes (e.g., Amphotericin B and Natamycin) and various azole derivatives as well as antibacterial agents, (e.g., polymyxin B), anti-viral agents and tranquilizing/anesthetic drugs such as benzodiazepines and anti-psychotic agents. Accordingly, another embodiment of the invention includes a composition for parenteral use including a water-insoluble/lipophilic-pharmaceutically-active agent and a first solvent, the first solvent including an alcohol and a lipid. The agent is dissolved in the first solvent. The composition optionally further includes a second solvent comprising an aqueous infusion fluid.

The invention also includes a method of preparing a water-insoluble/lipophilic pharmaceutically active agent for parenteral use by the steps of: 1) providing an aqueous lipid emulsion, 2) lyophilizing the aqueous lipid emulsion, 3) emulsifying the lyophilized emulsion in alcohol to produce a primary diluent, and 4) dissolving the pharmaceutically active agent in the primary diluent to produce a stock formulation. The aqueous lipid emulsion may be an aqueous lipid emulsion of soybean oil, the alcohol may be EtOH, and the agent may be chlorambucil. The method may further include the step of mixing the stock formulation with a second diluent, such as an aqueous infusion fluid. In addition to EtOH and soybean oil, other alcohols and lipids or acetic acid may be used to form the primary diluent without departing from the spirit and scope of the invention.

The invention also includes a method for treating a disease that is sensitive or responsive to chlorambucil treatment by parenterally administering a therapeutically effective amount of a chlorambucil composition to the patient. The chlorambucil composition may be any within the scope of the present invention as described above. Specifically, the chlorambucil composition may include chlorambucil; a first solvent, the first solvent including an alcohol and a lipid, where the chlorambucil is dissolved in the first solvent, and a second solvent including an aqueous infusion fluid. Diseases that may be treated include, but are not limited to, cancers, chronic lymphocytic leukemia, lymphoma, Hodgkin's disease, a myeloproliferative disorder, an autoimmune disease and transplant rejection. The chlorambucil composition may be administered intravascularly, intrathecally, subcutaneously, intramuscularly, or intraperitoneally, among other routes. After mixing with or suspending in a suitable ointment base, the composition may also be applied topically, such as in the treatment of a peripheral T-cell lymphoma. The patient can be any animal including mammals and humans.

The term “therapeutically effective amount” as used in this application means that a sufficient amount of the composition is added to achieve the desired therapeutic effect or another clinical effect, e.g., to slow down excessive skin cell proliferation in refractory psoriasis. The actual amount used will vary based on certain factors, such as the type of medical device used in administration, the age, sex, health, species and weight of the patient, and the use and length of use, as well as other factors known to those of skill in the art. Dose ranges are not limited and depend on individual characteristics of the patient and the specific disease entity that is the object of treatment.

Still another embodiment of the invention is directed to a method for parenterally administering chlorambucil to a patient by: 1) providing an aqueous lipid emulsion; 2) lyophilizing the aqueous lipid emulsion; 3) emulsifying the lyophilized emulsion in alcohol to produce a primary diluent; 4) dissolving the chlorambucil in the primary diluent to produce a stock formulation; 5) mixing the stock formulation with a second diluent to form an infusion fluid; and 6) administering the infusion fluid to the patient. The aqueous lipid emulsion may be an aqueous lipid emulsion of soybean oil and the alcohol may be EtOH. However, in addition to EtOH and soybean oil, other alcohols and lipids may be used to form the primary diluent without departing from the spirit and scope of the invention.

The following examples are included to demonstrate specific embodiments of the invention.

### EXAMPLES

### Example 1

### Chlorambucil Formulations Acceptable for Parenteral Administration

This example demonstrates the successful design of stable formulations of chlorambucil using solvent vehicles that are nontoxic and suitable for parenteral administration.

Preparation of Prototype Solvent Vehicle and Primary Stock Solution.

An EtOH/lipid/CLB solution (“primary stock solution”) as referenced in these Examples was prepared as follows. 10 ml of Intralipid™, an aqueous soybean oil emulsion available from Pharmacia (Peapack, N.J.) was lyophilized to remove all water. Subsequently, 2 g of the freeze-dried product was added to 10 ml of EtOH at room temperature and shaken vigorously for 1 minute to form an emulsion of lipid in ethanol, thereby forming the “prototype EtOH/lipid solvent vehicle.” Subsequently, 100 mg of CLB powder (Sigma, St. Louis, Mo.) was dissolved in approximately 1 ml of the prototype EtOH/lipid solvent vehicle to form the “primary stock solution,” having a final concentration of CLB in the solvent vehicle of 100 mg/ml.

Clinical studies of the anti-tumor or immune suppressive activities of CLB in humans have been conducted using the oral preparation, presently the only FDA-approved formulation. The previously utilized chlorambucil regimens typically prescribe a dose in the range of 4-12 mg orally once daily until clinical anti-tumor effect is obtained, or intermittent pulse doses of 20-100 mg/m² of body surface area (BSA) every 2-4 weeks. Several additional different dose regimens have been reported to be successful. The dose-limiting toxicity with the intermittent schedule appears to be neurological, including confusion and generalized seizure activity, usually manifested at doses of around 100 mg/m² BSA and above. The administration of low daily doses over prolonged time periods typically has not correlated with neurological adverse effects; instead, bone marrow suppression with a slow and insidious onset is typically dose limiting.

The potential danger of generalized seizure activity as a side effect of high-dose CLB administration mainly resides in the risk for trauma and bronchial aspiration of gastric content during the seizure.

A prolonged infusion schedule provides extended tumor cell drug exposure, while at the same time avoiding sudden high plasma peak concentrations that may trigger serious neurological side effects. From these observations it was determined that a stable formulation having a final concentration of CLB suitable for prolonged infusion would be desirable (e.g., final infusion fluid containing between 1-5 mg/ml). Chlorambucil has a short terminal half-life in blood, approximately 60 minutes when taken by mouth, and a prolonged infusion would extend drug exposure to the malignant tissues, yet decrease the plasma peak drug concentration that may be associated with serious neurological side effects.

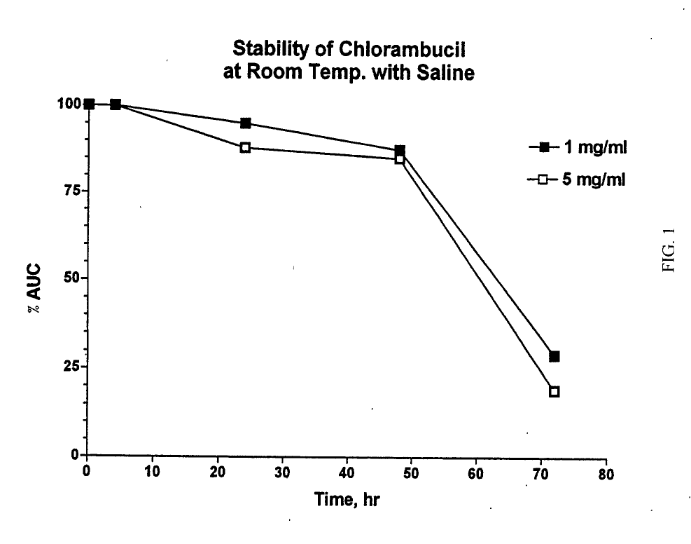

A solvent system of the present invention provides such a formulation that is stable (>90%) at room temperature (RT) for over 24 hours (see FIG. 1).

Specifically, CLB was dissolved in a solvent vehicle of EtOH/lipid (prototype EtOH/lipid solvent vehicle) and then diluted to appropriate concentrations with NS. Such systems are suitable for prolonged (≧12 hours) infusion time, yet their stability leaves an extensive margin of time for convenient handling in the pharmacy and on the medical floor prior to actual patient administration. Thus, if a clinical treatment dose of 1-5 mg/ml is desired, a stock formulation of between 10-25 mg/ml of CLB in EtOH/lipid could be easily diluted in NS or other infusion fluid to achieve such a final use-concentration. The clinician may then elect to infuse CLB over either short or prolonged time periods without having to exchange bags of infusate as might be needed if the formulation were unstable or subject to chemical degradation.

Enhanced Solubility in Physiologically Acceptable Solvents

The solubility of CLB was determined in several individual vehicles. Briefly, a known amount of CLB drug, formulated as a powder (Sigma, St Louis, Mo.), was equilibrated in the respective solvent at RT over 1-4 hours. An aliquot was removed, filtered, and diluted in methanol (MeOH) before high-pressure liquid chromatography (HPLC) to determine solubility at predetermined times. Based on the CLB solubility in each vehicle, different solvents were mixed according to the cosolvency principle in an attempt to enhance the stable solubility (Spiegel A. J., Noseworthy M. N.: Use of nonaqueous solvents in parenteral products. J. PHARM. SCI. 52:917-927, 1963; Yalkowsky S. H., Roseman T. J.: Solubilization of drugs by cosolvents. In: Yalkowsky S. H. (Ed.): TECHNIQUES OF SOLUBILIZATION OF DRUGS. pp. 91-134. Marcel Dekker Inc., New York, N.Y. 1981).

Different solvent systems were evaluated relative to estimates to arrive at a stable stock formulation that would be useful in the clinical situation. An assumption was that intermittent “high-dose” administration or a prolonged infusion would be the preferred modes of administration, i.e., the infusion schedule would require the solvent vehicle to accept a high dissolved drug concentration. The stock formulation was then diluted with a final solvent to yield a stable formulation. Between 1-5 mg/ml was the desired range of the formulation, as it could be conveniently infused intravascularly.

Several water-miscible, physiologically-acceptable vehicles that would be compatible with human administration were examined. The candidate solvents included DMSO, polyethylene glycol-400 (PEG), propylene glycol (PG), in addition to the aqueous solvents NS, 5% dextrose in water and soybean lipid emulsion (Intralipid™, Pharmacia, Peapack, N.J.). DMSO and EtOH were the best primary solvents, whereas CLB was virtually insoluble in the aqueous solvents. When the drug was dissolved in DMSO, it started degrading within a few hours in the solvent (data not shown). Further, there was concern that the sulfur moiety of DMSO could be chemically reactive with CLB. Ultimately, a solvent vehicle composed of EtOH and emulsified soybean oil was discovered (the prototype EtOH/lipid solvent vehicle, described above). This solvent vehicle allowed solubility at CLB concentrations of at least 100 mg/ml, and the drug remained stable in solution for over 24 hours at RT (FIG. 1).

HPLC Assay

HPLC assay provides an accurate and sensitive detection system for low concentrations of CLB in solution, both protein-free mixtures and protein-containing fluids (i.e., blood plasma), utilizing absorbency detection in the ultraviolet (UV) spectrum. For the detection a wavelength of 254 nm was chosen on the basis of the inherent absorption maximum of the CLB molecule. The HPLC system was equipped with a Waters 717 plus AutoSampler (Waters, Milford, Mass.). The detector was a Waters Model 486 Tunable Absorbance Detector in sequence with a Waters Millennium™ software package for HPLC (Waters, Milford, Mass.). The column used was a Whatman C-18 EQC 10 μL 125A μBondapak 4.6×216 mm. (Whatman, Springfield Mill, Kent, Maidstone, UK) The isocratic mobile phase consisted of 35% acetonitrile and 65% sodium acetate buffer (pH 3.8), which was prepared from acetic acid (0.2 N) and a solution of sodium acetate (55 g/liter). A total of 440 ml acetic acid and 60 ml sodium acetate solution was poured into a 1-liter flask, and deionized water was added up to the desired volume marker. All chemicals were HPLC grade unless otherwise indicated. The mobile phase flow rate was 1.5 ml/min. The analytic system was based on previously established extraction and HPLC experience with retinoic acid and with the bifunctional DNA-alkylating drug busulfan (Napoli J L, Pramanik B C, Williams J B et al: Quantification of retinoic acid by gas-liquid chromatography-mass spectrometry: total versus all-trans-retinoic acid in human plasma. J LIPID RES. 26: 387-392, 1985; Chow D S-L, Bhagwatwar H P, Phadungpojna S, Andersson B S: Stability-indicating high-performance liquid chromatographic assay of busulfan in aqueous and plasma samples. J CHROMATOGR B, 704: 277-288, 1997).

To avoid analytical interference from endogenous plasma proteins in the chromatogram when assaying CLB in plasma samples, an extraction/purification step utilizing disposable OASIS™ HEL extraction cartridges (Waters Corporation Inc., Milford, Mass.) was added. Briefly, these disposable extraction columns were conditioned with 1 ml MeOH and 1 ml water. One milliliter of plasma was then loaded on the column, and after the plasma was washed once with 1 ml of 5% MeOH in water and twice with 1 ml of “acidic MeOH in water” (2% acetic acid and 5% MeOH in water), elution was performed (65% MeOH in H₂O with 2% NH₄OH). The elute was then analyzed by HPLC using the isocratic mobile phase described above.

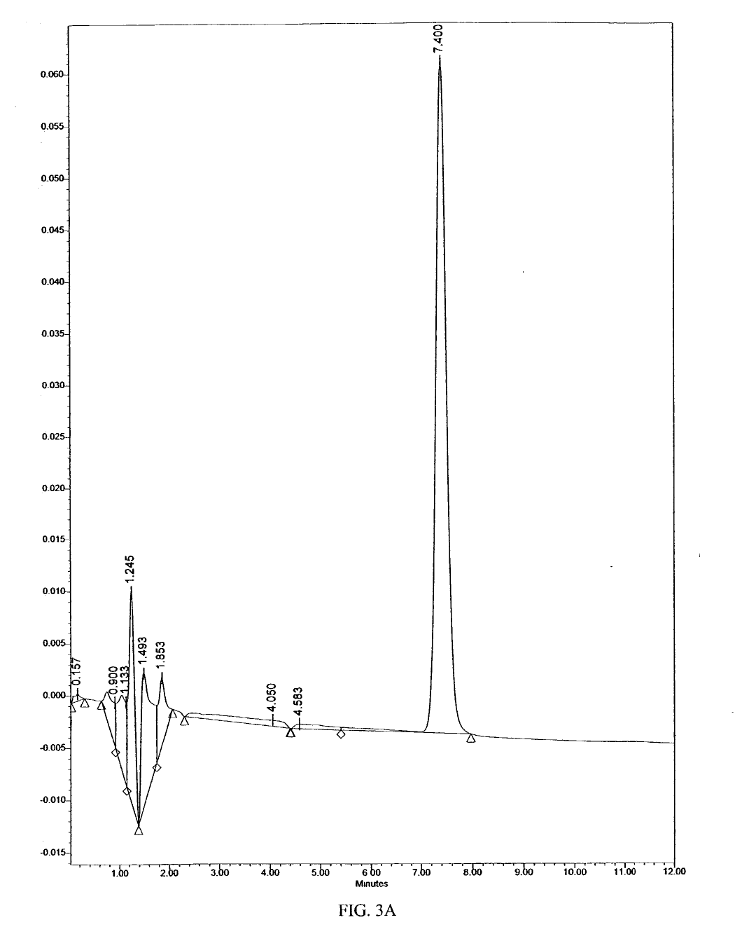

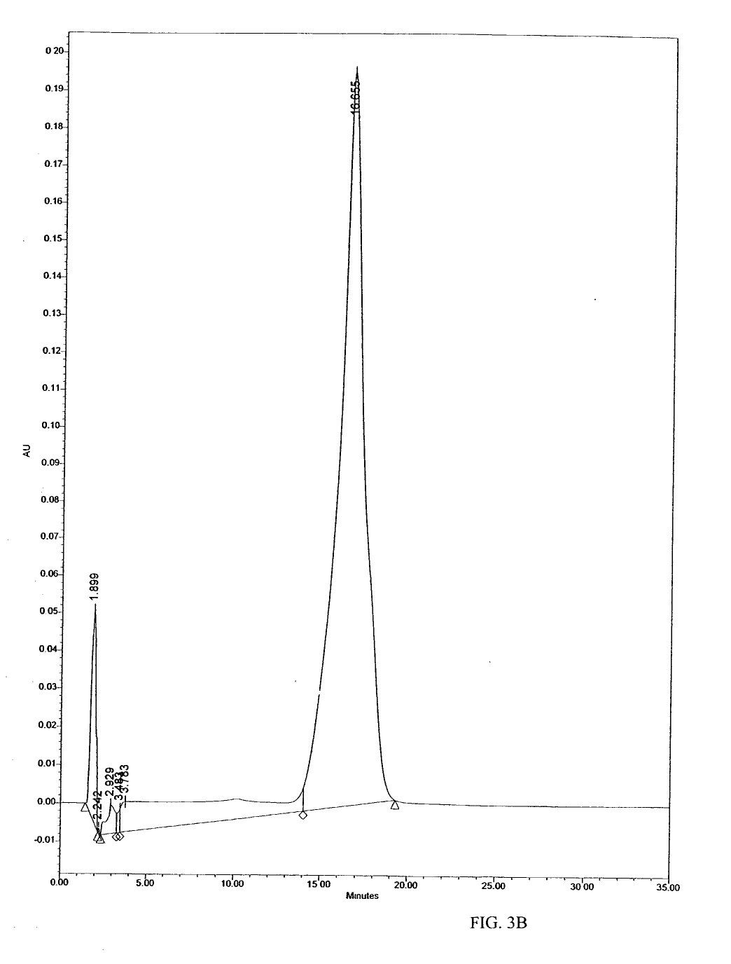

Examples of CLB chromatograms from the HPLC assay are shown in FIGS. 3(a) and (b). In (a), the Whatman C-18 EQC 10 μL 125 A μBondapak column was used. The injected sample volume (100 μg/ml) was 200 μl. In (b), the C₈Symmetry™ column was used and the injected volume was 20 μl (the injected concentration was 100 μg/ml).

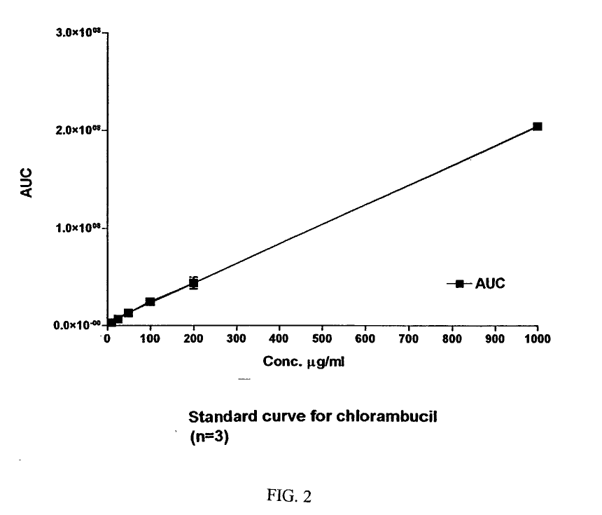

In the sampler analyzed in FIGS. 3a and 3b the CLB was dissolved in EtOH/lipid (prototype EtOH/lipid solvent vehicle) and further diluted to 5 mg/ml and 1 mg/ml, respectively, using normal saline as the final solvent. The HPLC retention time under the above conditions utilizing the Symmetry C8 column was about 18-20 min. The Whatman C-18 EQC 10 μL 125A μBondapak column yielded a retention time of only 11-12 min. The assay was linear from 1 μg/ml to 1,000 μg/ml in protein-free solutions, i.e., the various solvent systems utilized in the formulation-feasibility and -stability studies (FIG. 2). FIG. 2 is the standard curve of chlorambucil concentration vs. area under the curve for the high-pressure liquid chromatography (HPLC) assays described above. The x-axis shows concentration in μg/ml, and the y-axis shows the AUC.

This HPLC assay consistently yielded high recovery and accuracy and a lower sensitivity limit of about 1 μg/ml. By increasing the volume injected in the HPLC beyond 20 μl, the lower sensitivity limit was reproducibly improved to 5-10 ng/ml. This HPLC technique was standardized and used for all the stability studies without additional modifications.

### Example 2

### Demonstration of In vitro Stability and Other Properties of One of the Novel Formulations

In this example, a stable CLB formulation suitable for human administration was evaluated.

Solubility Studies

An excess of CLB as a powder was added to DMSO, PEG, and PG at RT. Each mixture was placed in a dark environment and checked visually for up to 4 hours for evidence of solubilization. Samples of 1 ml were taken at various time intervals. The CLB concentration was then determined by HPLC after filtration through a 0.45 μm polytetrafluoroethylene (PTFE) membrane filter (Autovial®) fitted to a syringe assembly (Whatman Inc., Springfield Mill, Maidstone, Kent, UK).

A maximum equilibrium CLB concentration of>100 mg/ml was achieved in DMSO and EtOH within 1 hour at room temperature (RT). PEG-400, PG, NS, 20% soybean lipid emulsion (Intralipid™), and 5% dextrose did not yield any significant concentrations of solubilized drug. The latter were therefore not considered for further study as primary solvents. In DMSO, the CLB was stable for at most 2 hours at RT before significant degradation occurred. Additionally, there was concern that the sulfur group of DMSO could react with CLB upon extended exposure. An EtOH/lipid solvent (prototype EtOH/lipid solvent vehicle) was identified as the preferred primary solvent vehicle for the continued investigations.

At a CLB concentration of 100 mg/ml in the EtOH/lipid solvent (prototype EtOH/lipid solvent vehicle), the CLB was stable for more than 7 days at RT. When primary stock solution of EtOH/lipid/CLB was diluted with NS to 1 mg/ml or 5 mg/ml respectively, CLB was still stable with>95% recovered at 24 hours (FIG. 1, FIG. 3). Based on these findings, it was determined that CLB is stable enough for clinical use in this solvent vehicle.

Stability of the Various CLB Formulations

The physical and chemical stability of the various intended parenteral formulations were studied as follows, using the EtOH±lipid (Intralipid™) formulation as an example:

CLB was dissolved at a concentration of 100 mg/ml in EtOH only or in EtOH/lipid (prototype EtOH/lipid solvent vehicle) and incubated at 4° C. and at 22° C. The resulting CLB concentration was measured by HPLC in samples taken immediately after solubilization, then hourly for 8 hours, and then at gradually increasing time intervals for up to 7 days, depending on the initial rate of solubilization/degradation in the respective solvent system.

The CLB solubility differed markedly between different primary solvents. A solubility in excess of 100 mg/ml was reached using DMSO and the ethanol/lipid formulation (the prototype EtOH/lipid solvent vehicle). The favored primary solvent vehicle investigated further in the extended studies was the ethanol/lipid formulation (prototype EtOH/lipid solvent vehicle), as this formulation did not appear to have any discernible CLB degradation even over extended time (seven days) at RT. In contrast, although DMSO provided excellent solubility of CLB, the drug started degrading within a few hours. It was hypothesized that because CLB is very lipophilic, the combination of a lipid emulsion and EtOH would render the subsequent dilution in a purely aqueous vehicle (NS or 5% dextrose) possible without precipitation or rapid chemical degradation. The CLB “stock” concentration in this composite solvent could be kept at least as high as 100 mg/ml, with a resulting low overall EtOH concentration after dilution to a desired concentration of 1-5 mg/ml (FIG. 1, FIG. 3). The hemolytic potential for the final formulation should be minimal, and it should also yield negligible amounts of EtOH to the recipient. Even at hypothetical clinical CLB doses of 100-150 mg/m² BSA, the patient's total EtOH dose would be around 2.0-3.5 grams.

In summary, the stability of CLB in the favored EtOH/lipid solvent system (prototype EtOH/lipid solvent vehicle) was excellent: at 7 days>95% of the drug was still intact at RT as assayed by HPLC.

Osmotic Pressure Measurement

It is desirable that a parenteral formulation of a pharmacologically active agent be isosmotic with blood. A hypertonic delivery system can be utilized if the drug formulation is infused through an indwelling (central) venous catheter and gradually diluted in a large blood volume. Osmotic pressures of an exemplary formulation (prototype EtOH/lipid solvent vehicle±CLB) are shown in Table 1. Osmotic pressures were measured with a micro-osmometer model 3MOplus osmometer (Advanced Instruments Inc., Needham Heights, Mass.). The instrument was calibrated using Advans™ intrinsic calibration standards (Advanced Instruments Inc.) in the range of 500-2000 mOsm/kg. The test solution was placed in a disposable cuvette, and the osmotic pressure readings were recorded. Triplicate measurements were carried out for each vehicle (without CLB), and six measurements were done with drug added. A two-tailed t-test was used to compare the differences in osmotic pressures of various solvent vehicles with and without the addition of CLB (Mann H. B., Whitney D. R.: On a test whether one of two random variables is stochastically larger than the other. ANN. MATH. STATIST. 18: 50-60, 1947). The difference between the means of the two groups is considered significantly different for P<0.05.

As shown in Table I, the ethanol/lipid stock vehicle (prototype EtOH/lipid solvent vehicle) without (or with) CLB was hypertonic; its osmotic pressure was estimated at more than 2322 mOsm/kg, as compared with 280-295 mOsm/kg for human blood. In contrast, the mixture of EtOH/lipid/CLB further diluted in NS rapidly approached isosmolarity. The osmolarity of this complete vehicle was not appreciably changed by the addition of CLB at a final concentration of 1-5 mg/ml (<5%).

TABLE I
OSMOTIC PRESSURES OF FORMULATION OF THE
PRESENT INVENTION WITH AND
WITHOUT CHLORAMBUCIL

| | | Osmotic |
|---|---|---|
| | Number of | Pressure |
| | Independent | mosm/kg |
| Solution | Observations | (±S.D.) |
| Water | 3 | 42 |
| Saline | 3 | 233.0 |
| Blood | 3 | 280-295 |
| Intralipid ™ | 3 | 373 (±9) |
| (20%) | | |
| EtOH | 3 | 2445 |
| Vehicle | 3 | 2384 |
| Intralipid ™ | | (±6) |
| (20%) | | |
| EtOH | | |
| Chlorambucil | 3 | 2322 |
| EtOH | | (±7) |
| Intralipid ™ | | |
| (20%) | | |

Hemolysis Studies In Vitro

The procedure of Parthasarathy et al. was used to examine the hemolytic potential of a few selected formulations of the present invention, and the LD₅₀ (concentration that results in 50% lethality or destruction of a population) values of the most optimal formulation was constructed as previously described (Parthasarathy R, Sacks P G, Harris D, et al: Interaction of liposome-associated all-trans-retinoic acid with squamous carcinoma cells. CANCER CHEMOTHER. PHARMACOL. 34:527-34, 1994). Briefly, heparinized blood was mixed with an equal volume of Alsever's solution. This mixture was washed twice in phosphate-buffered saline (PBS), and a 10% (volume per volume, v/v) erythrocyte/PBS solution was then prepared and mixed with increasing amounts of the solvent (prototype EtOH/lipid solvent vehicle and NS) with or without addition of CLB. These resulting mixtures were incubated for 4 hours at 37° C. At the end of the incubation, the cells were pelleted at 10,000×g in an Eppendorf micro-centrifuge, and after washing twice in NS, the pellet was resuspended and lysed using distilled water. The release of hemoglobin in the supernatant (i.e., hemolysis) was determined spectrophotometrically at a wavelength of 550 nm. Maximum lysis was measured against a reference erythrocyte solution that had been lysed by hypotonic shock.

The hemolytic potential of the formulations (EtOH/lipid/CLB/NS) were evaluated as described. The results were plotted as the fraction of intact erythrocytes versus concentration (total volume percent) of the solvent vehicle. The total volume percent was defined as the volume percent of the solvent system in the mixture after addition of the erythrocyte suspension. This was done to simulate the dilution of the drug formulation in the blood stream after parenteral administration. Intact, healthy erythrocytes were defined as those capable of retaining their hemoglobin intracellularly after mixture with the solvent vehicle with or without CLB.

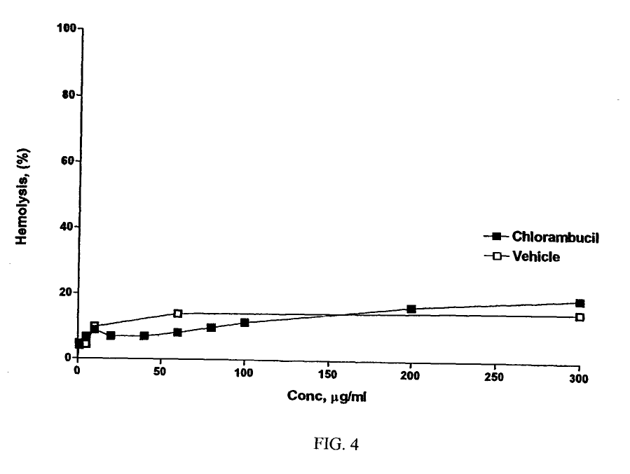

As shown in FIG. 4, the formulation tested showed a very low tendency to induce hemolysis when the formulation complete vehicle (prototype EtOH/lipid solvent vehicle and NS) was used either with or without the addition of CLB. The CLB-dependent lysis barely exceeded background for drug concentrations of 100 μg/ml or more.

In conclusion, the EtOH/lipid/NS formulation had very low hemolytic potential and should be completely safe for (human) intravascular (and also intrathecal) administration.

In Vitro Cytotoxicity of CLB

The cytotoxic potential of selected solvent systems with and without CLB was determined against the two human myeloid leukemia cell lines (HL-60) (Gallagher R. Collins S, Trujillo et al: Characterization of the continuously differentiating myeloid cell line (HL-60) from a patient with acute promyelocytic leukemia. BLOOD 54:254-68, 1979) and human myeloid leukemia cell line (KBM-7/B5) (Andersson B S, Beran M, Pathak S, et al: Ph-positive chronic leukemia with near-haploid conversion in vivo and establishment of a continuously growing cell line with similar cytogenetic pattern. CANCER GENETICS AND CYTOGENET 24:335-43, 1987; Andersson B S, Collins V P, Kurzrock R, et al: KBM-7; human myeloid leukemia cell line with double Philadelphia chromosomes but lacking normal BCR and c-ABL transcripts. LEUKEMIA, 9:2100-2108, 1995). Briefly, HL-60 or KBM-7/B5 cells were suspended at a density of 5,000 cells per ml in Iscove's modified Dulbecco medium (IMDM) supplemented with 20% fetal bovine serum (FBS) and 0.3% agar as viscous support. Chlorambucil in the prototype EtOH/lipid solvent formulation, further diluted with NS (1:10, v/v), was added to achieve final EtOH concentrations of<1%, at increasing concentrations of EtOH/lipid (0.5%, 1.0%, 2.0%, 3.0%, and 10%, v/v). The final CLB concentrations ranged from 0.1 to 10.0 μg/ml. After gelling of the agar at RT, the cells were incubated for 7-8 days at 37° C. in a fully humidified atmosphere of 5% CO₂ in air. Clones of>50 cells were counted under an inverted phase contrast microscope, and survival curves were constructed as described (Andersson B S, Mroue M, Britten R A, Murray D: The role of DNA damage in the resistance of human chronic myeloid leukemia cells to cyclophosphamide analogs. CANCER RESEARCH 54:5394-5400, 1994).

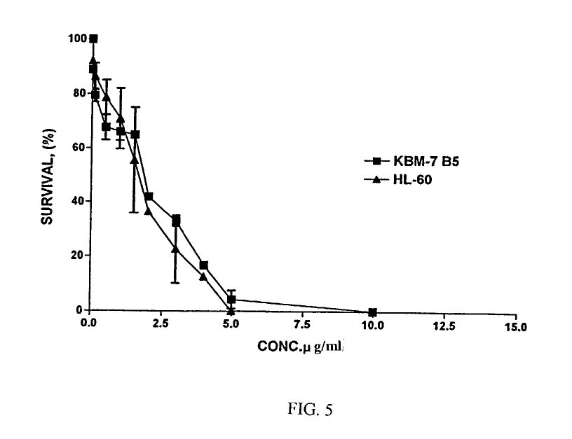

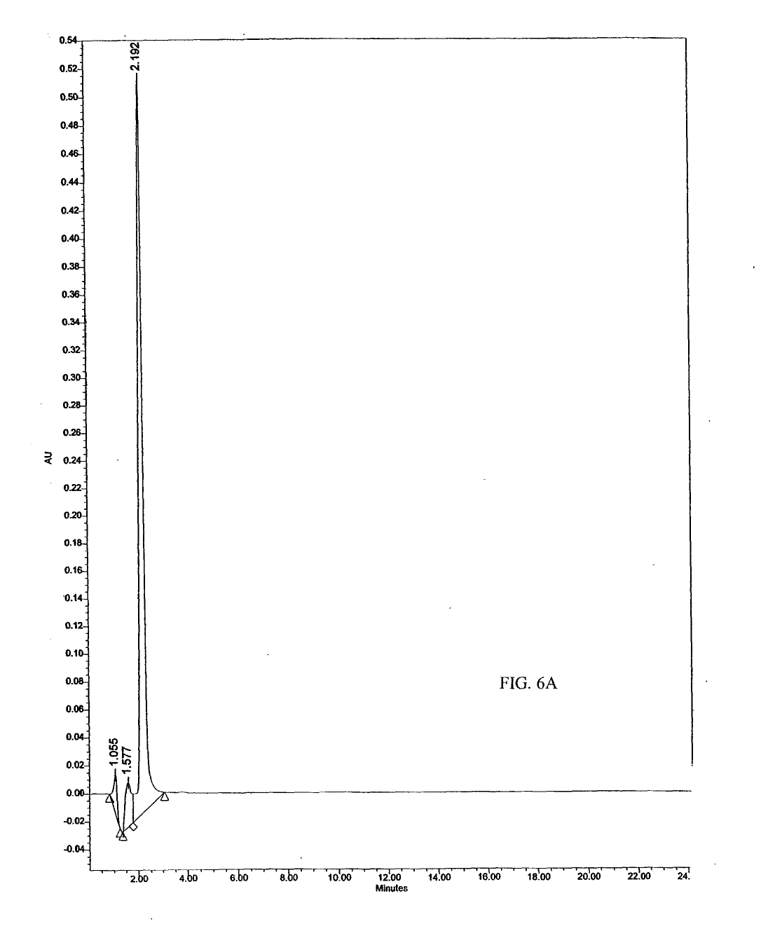

The HL-60 and KBM-7/B5 myeloid cells were exposed to CLB in the formulation of EtOH/lipid/NS at increasing volume ratios of up to 5%; cell cultures exposed in parallel to the complete formulation (EtOH/lipid/NS) alone served as a negative control. The examined solvent system (at a volume ratio of up to 5%) did not exhibit any detectable toxicity in the concentrations achieved at the highest tested CLB concentrations against either cell line in these experiments (not expressly shown). When the complete CLB formulation was added in increasing concentrations to the cells, concentration-dependent cytotoxicity was apparent (FIG. 5). Chlorambucil retained full cytotoxic activity in the formulation when tested in parallel with cells exposed to CLB dissolved in acetone as a positive control.

### Example 3

### Quantitative CLB Analysis in Plasma and Pharmacology of IV CLB

This example demonstrates that CLB in a formulation of the present invention mixed with blood or plasma may be recovered as native drug using quantitative extraction technology and HPLC assay, and that the CLB concentrations remain in the cytotoxic range for several hours after IV administration. It further shows that the plasma pharmacokinetics after parenteral administration of CLB in a formulation of the present invention to rats and dogs conforms to what can be expected based on the published pharmacology of oral CLB.

Quantitative Extraction of Chlorambucil in Plasma

One milliliter each of human, dog, and rat plasma was mixed with various amounts of the reformulated CLB (<3% of the final volume) to yield a drug concentration of 0.05-3.0 μg/ml (the formulation EtOH/lipid/NS/CLB was made from the prototype EtOH/lipid solvent vehicle and NS, having a CLB concentration 10 mg/ml). The drug was then extracted from the plasma samples and analyzed by HPLC as described in Example 1. Briefly, 1 ml plasma was eluted on the OASIS™ HEL extraction cartridge using 65% methanol (MeOH) in H₂O with 2% NH₄OH as the eluting phase. The resulting elute was analyzed by HPLC with CLB detected spectrophotometrically at 254 nm. The drug recovery from rat plasma spiked to 10 μg/ml was calculated to be 87±3%. The assay was linear in the interval from 10 ng/ml to 100 μg/ml, with a detection limit of 5 ng/ml.

Parenteral CLB: Experimental Protocol in Rats and Beagle Dogs

Male Sprague-Dawley rats with a body weight of 250-300 g were used for the in vivo pharmacology experiments (Harlan-Sprague-Dawley, Houston, Tex.). The animals were allowed a minimum of 3-4 days after arrival to accommodate to the new environment and allowed free access to commercial feed and tap water prior to and during the experimentation period. The animals were housed in facilities that meet the requirements of the U.S. Department of Agriculture, (USDA), National Institute of Health (NIH), and Department of Health and Human Services (DHHS).

The CLB dose of 5 mg/kg BW was determined to be the highest dose that could be administered to the rats as an IV bolus injection without requiring anticonvulsant premedication.

The CLB was formulated in EtOH/lipid (prototype EtOH/lipid solvent vehicle) to a stock concentration of 100 mg/ml and then diluted with NS so the dose (5.0 mg/kg) could be injected in a tail vein in a volume of approximately 0.5 ml. The CLB concentrations of the formulation were confirmed by HPLC prior to all administrations. No anticonvulsant premedication for the rats in this experiment was used in order to avoid the possible induction of microsomal liver enzymes that could modify CLB metabolism. The animals were unanesthetized, being only physically restrained during the drug injection for the same reason.

The CLB given to beagle dogs was prepared as above. The same general design of the experiment was used, except that the dogs received a maximum dose of 7.5 mg/kg BW through a cephalic vein catheter, and the drug was delivered under general anesthesia. Due to a generalized seizure after CLB administration in the first dog, two subsequent animals were premedicated with diphenylhydantoin as a prophylactic measure, and they tolerated the CLB administration without any obvious neurological effects.

Blood samples (0.5-1.0 ml) were drawn in heparinized tubes at selected time points prior to the drug infusion (“blank”), and from 5 min to 6 hours after drug injection for determination of CLB concentrations. The samples were obtained through cardiac puncture under light CO₂ anesthesia in the rats and through a prepositioned cephalic vein catheter in the dogs. The blood was centrifuged at 1,000×g for 10 min, and the plasma was removed and stored at−80° C. until extracted and assayed by HPLC.

Chlorambucil in Plasma and IV Drug Pharmacology Results

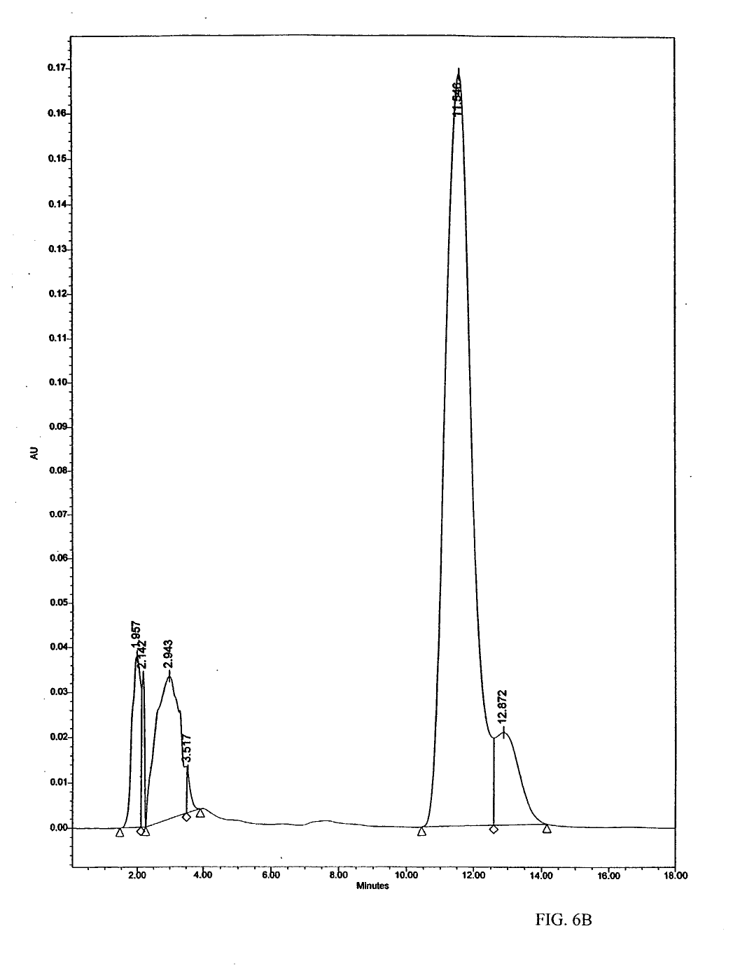

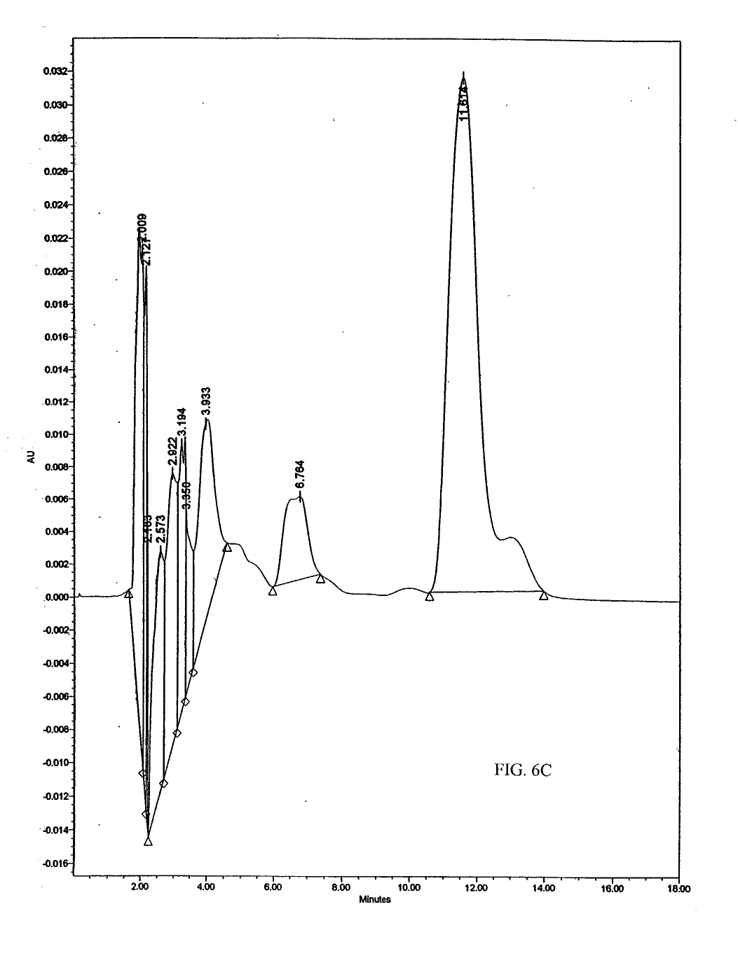

The drug extraction from plasma using the OASIS HEL 1 ml extraction cartridges was essential to avoid interference from endogenous plasma protein components and thus to recover the maximum amount of drug. Authentic chromatograms from blank plasma FIG. 6(a), CLB-spiked plasma FIG. 6(b), and one plasma sample obtained from the current pharmacokinetic study FIG. 6(c) are shown.

The CLB retention time in this system was 11.3-12.3 min, when using the Whatman 125A μBondapak column (see Example 1). The recovery of CLB with the described technique was 87±3% from rat plasma spiked in vitro with 10 μg/ml of drug. The assay was linear after drug extraction from plasma samples spiked in the concentration range from 10 ng/ml to 50 μg/ml. The limiting sensitivity was about 5 ng/ml, when 200 μl was injected into the chromatograph. This could be improved by utilizing a larger part of the 2 ml injection loop in the system, and also by evaporating/reconstituting the 500 μl elute in a smaller volume to obtain a higher CLB concentration in the injected sample. A standard curve was prepared in the range from 10 μg/ml to 1,000 μg/ml for the pharmacology experiment (not shown), and a good linear correlation (r=0.9999) was obtained between the actual plasma CLB concentrations and the measured AUC values.

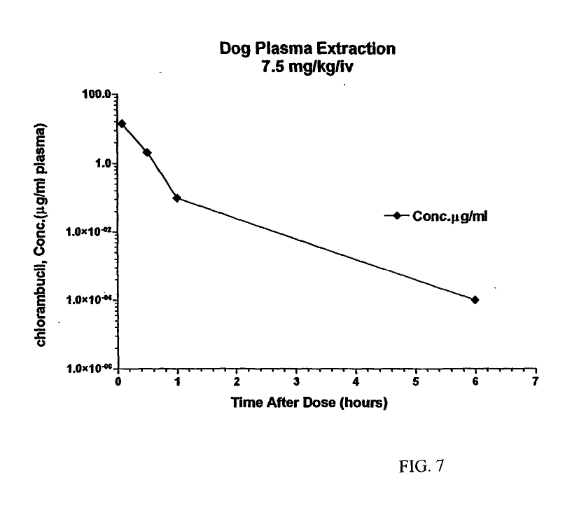

The resulting data illustrate that the utilized novel CLB formulation produces detectable, cytotoxic CLB plasma concentrations after injection of 5 mg/kg BW in rats and 7.5 mg/kg BW in beagles (FIG. 7). FIG. 7 is a graph showing the change in plasma concentration over time of 7.5 mg/kg CLB injected into a beagle dog. The apparent terminal half-life of CLB is in the range of 60 minutes under the conditions used with this formulation.

The injections were well tolerated, but the higher dose caused seizures, similar to what has been described using oral high-dose CLB in humans. (Johnson S, Smith A G, Loffler H: Multicentre prospective randomized trial of fludarabine versus cyclophosphamide, doxorubicin, and prednsione (CAP) for treatment of advanced-stage chronic lymphocytic leukemia. The French Cooperative Group on CLL. LANCET 347: 1432-1438, 1996).

In summary, the data prove that novel pharmaceutically acceptable, stable formulations of chlorambucil of the invention can be safely utilized for intravascular administration. An exemplary solvent vehicle is physiologically compatible with intravascular administration and was used as an example to demonstrate in both rodent and beagle models that the injection of CLB in this formulation was well accepted and had insignificant acute solvent system toxicity. The injection of this formulation in rats (5.0 mg/kg BW) and beagles (7.5 mg/kg BW) yielded CLB plasma concentrations that for several hours remained in the cytotoxic range.

The data for the formulation used in these examples, EtOH/lipid/NS/CLB, conclusively prove that it is now feasible to introduce CLB for parenteral administration in clinical therapy of malignant and autoimmune diseases. This can be expected to result in the predictable and reproducible attainment of greatly improved cytotoxic/immunosuppressive activity. These results can also provide a reasonable expectation of insignificant normal organ toxicity from the solvent vehicle. In particular, it is possible and in fact likely that serious hypersensitivity reactions may be completely avoided with this formulation.

The novel formulations of the present invention improve the clinical safety of CLB-based therapy and make it possible to optimize the use of this important drug in the treatment of cancer and autoimmune disorders. Embodiments of the invention may also be used in combination chemotherapy for conditioning of patients undergoing hemopoietic stem cell transplantation.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention.

All references cited herein, to the extent that they provide exemplary, procedural or other details supplementary to those set forth herein, are specifically incorporated herein by reference.

### ABBREVIATIONS USED IN THIS APPLICATION

AUC—area under the curve, term used to denote the actual measured area of a peak in a chromatogram.

BSA—Body surface area.

BW—Body weight.

CLL—Chronic lymphocytic leukemia

CLB—Chlorambucil.

DMF—Dimethylformamide.

DMSO—Dimethylsulfoxide.

EtOH—Ethanol.

FBS—Fetal bovine serum.

HL-60—Human myeloid leukemia cell line.

IMDM—Iscove's modified Dulbecco medium (GIBCO, Grand Island, New York, N.Y.).

Intralipid™—Brand name of an aqueous lipid emulsion made primarily from soybean oil and marketed for parenteral nutrition available from The soybean lipid emulsion was freeze-dried before use as a solvent in the ensuing studies and is referred to as “lipid” in this text.

KBM-7/B5—Human myeloid leukemia cell line.

LD₅₀—The concentration or dose that results in 50% lethality or destruction of a population.

Liposyn™—Brand name of an aqueous lipid emulsion made primarily from soybean oil and marketed for parenteral nutrition available from Abbott (Abbott Park, Ill.). The soybean lipid emulsion was freeze-dried before use as a solvent in the ensuing studies and is referred to as “lipid” in this text.

MeOH—Methanol.

MTT—3, [4,5-Dimethylthiazol-2-yl] 2,5-diphenyltetrazolium-bromide.

NH₄—acetate—Ammonium acetate.

NS—Normal saline (150 mM NaCl).

OASIS™ HLB—Disposable extraction cartridges from Waters Inc., Milford, Mass.

PBS—Phosphate-buffered saline (Dulbecco's formulation, pH 7.4).

PEG—Polyethylene glycol-400.

PG—Propylene glycol.

PTFE—Polytetrafluoroethylene (filters), Teflon.™

RT—Room temperature (22° C.).

SDS—Sodium dodecylsulphate.

What is claimed is:

1. A composition for parenteral use comprising:
chlorambucil; and
a first solvent including an alcohol and a lipid, wherein the chlorambucil is dissolved in the first solvent.

2. The composition of claim 1 further comprising the composition stable for at least seven days at room temperature.

3. The composition of claim 1 wherein the first solvent comprises an emulsion of ethanol and soybean oil.

4. The composition of claim 3 wherein the soybean oil comprises a freeze-dried, aqueous soybean oil emulsion.

5. The composition of claim 3 wherein the soybean oil comprises an aqueous lipid emulsion selected from the group consisting of Intralipid™ or Liposyn™.

6. The composition of claim 3 wherein the ethanol comprises between 5 and 99% of the first solvent and the soybean oil comprises between 1 and 95% of the first solvent.

7. The composition of claim 1 further comprising a second solvent including an aqueous infusion fluid.

8. The composition of claim 7 further comprising the infusion fluid selected from the group consisting of normal saline, dextrose in water, and a lipid-based infusion emulsion fluid.

9. The composition of claim 1 wherein the composition comprises between 10 mg/ml to 25 mg/ml of chlorambucil prior to dilution in a secondary solvent.

10. The composition of claim 7 wherein the second solvent comprises normal saline and the composition comprises between 1 mg/ml and 5 mg/ml chlorambucil.

11. The composition of claim 10 further comprising the composition stable for at least twelve hours at room temperature.

12. The composition of claim 1 further comprising the parenteral use selected from the group consisting of intravascular, intrathecal, subcutaneous, intramuscular, and topical administration.

13. A composition for parenteral use comprising:
a water-insoluble/lipophilic pharmaceutically-active agent; and
a first solvent including an alcohol or an acid and a lipid, wherein the agent is dissolved in the first solvent.

14. The composition of claim 13 further comprising a second solvent including an aqueous infusion fluid.

15. The first solvent of claim 13 wherein the acid is an organic acid.

16. The composition of claim 13 further comprising the parenteral use selected from the group consisting of intravascular, intrathecal, subcutaneous, intramuscular, and topical administration.

17. A method for preparing a water-insoluble/lipophilic pharmaceutically-active agent for parenteral use comprising:
lyophilizing an aqueous lipid emulsion;
emulsifying the lyophilized emulsion in an alcohol to produce a primary diluent; and
dissolving the pharmaceutically-active agent in the primary diluent to produce a stock formulation.

18. The method of claim 17 wherein the agent is chlorambucil, the lipid is soybean oil and the alcohol is ethanol.

19. The method of claim 17 further comprising mixing the stock formulation with a second diluent.

20. The method of claim 19 wherein the second diluent is an aqueous infusion fluid.

21. A method for treating a disease responsive to chlorambucil comprising:
dissolving chlorambucil in a first solvent to create a first diluent of chlorambucil;
the first solvent including an alcohol and a lipid;
dissolving the first diluent of chlorambucil in a second solvent to create a second diluent of chlorambucil;
the second solvent including an aqueous infusion fluid; and
parenterally administering to a patient a therapeutically effective amount of the second diluent of chlorambucil.

22. The method of claim 21 wherein the disease is selected from the group consisting of chronic lymphocytic leukemia, lymphoma, Hodgkin's disease, a myeloproliferative disorder, an autoimmune disease, psorasis and transplant rejection.

23. The method of claim 21 wherein said disease is cancer.

24. The method of claim 21 wherein the patient is a human.

25. The method of claim 21 wherein the second diluent of chlorambucil composition is administered by a mode selected from the group consisting of intravascular, intrathecal, subcutaneous, intramuscular, or topical administration.

26. A method for parenterally administering chlorambucil to a patient comprising:
lyophilizing an aqueous lipid emulsion;
emulsifying the lyophilized emulsion in an alcohol to produce a primary diluent;
dissolving the chlorambucil in the primary diluent to produce a stock formulation;
mixing the stock formulation with a second diluent to form an infusion fluid; and
administering the infusion fluid to the patient.

27. The method of claim 26 wherein the patient is a human.

28. The method of claim 26 wherein the lipid is soybean oil and the alcohol is ethanol.

29. The composition of claim 28 wherein the ethanol comprises between 5 and 99% of the first solvent and the soybean oil comprises between 1 and 95% of the primary diluent.

30. A method for parenterally administering chlorambucil to a patient comprising:
lyophilizing an aqueous lipid emulsion;
emulsifying the lyophilized emulsion in an organic acid to produce a primary diluent;
dissolving the chlorambucil in the primary diluent to produce a stock formulation;
mixing the stock formulation with a second diluent to form an infusion fluid; and
administering the infusion fluid to the patient.

31. The method of claim 29 wherein the organic acid is acetic acid.
